(19) United States
(12) Patent Application Publication
(10) Pub. No.: US 2026/0171254 A1
(43) Pub. Date: Jun. 18, 2026

(54) Envirogenic Risk Score: Integrating Environmental and Genetic Factors for Enhanced Disease Risk Prediction

(71) Applicant: Reagan Moseti Mogire, Rockville, MD (US)

(72) Inventor: Reagan Moseti Mogire, Rockville, MD (US)

(21) Appl. No.: 18/981,577

(22) Filed: Dec. 15, 2024

(51) Int. Cl. G16H 50/30; G16B 20/20; G16B 40/00; G16H 10/20; G16H 10/60; G16H 80/00

(52) U.S. Cl. CPC G16H 50/30; G16B 20/20; G16B 40/00; G16H 10/20; G16H 10/60; G16H 80/00

(57) ABSTRACT

The present invention provides a comprehensive method and system for predicting phenotypes of individuals by integrating genetic data, environmental factors, and their interactions. Utilizing advanced statistical analyses, machine learning techniques, and computational models, the invention enhances the accuracy of phenotype prediction beyond traditional methods that consider genetic or environmental factors in isolation. This integrated approach supports personalized healthcare strategies, research initiatives, and public health policies. Key features include the collection and processing of genetic and environmental data, application of predictive algorithms incorporating weights and interaction terms, and generation of phenotype predictions accompanied by confidence scores. The system architecture encompasses secure databases, processors for complex computations, output devices for result presentation, and user interfaces for data input and user interaction. Adaptable across various platforms, the invention applies to a wide range of phenotypes, including disease risk, physiological traits, and behavioral characteristics, thereby advancing personalized medicine and contributing to improved health outcomes.

### CROSS-REFERENCE TO RELATED APPLICATIONS

This application claims the benefit of U.S. Provisional Patent Application No. 63/566,968, filed on Mar. 19, 2024, entitled “Integrating Environmental and Genetic Factors for Enhanced Disease Risk Prediction,” which is incorporated herein by reference in its entirety.

### STATEMENT REGARDING FEDERALLY SPONSORED RESEARCH OR DEVELOPMENT

This invention was not made with any federal funding, nor was it sponsored by any federal agency. Accordingly, the United States Government does not have any rights in this invention.

### FIELD OF THE INVENTION

The present invention pertains to the fields of biomedical informatics, computational biology, and personalized medicine. Specifically, it relates to systems and methods for calculating integrated disease risk scores by combining genetic data, environmental factors, and their interactions to enhance disease risk prediction. The invention leverages advances in genomics, epidemiology, and data science to provide a comprehensive assessment of an individual's susceptibility to complex diseases. It addresses the need for more accurate predictive models in healthcare by integrating diverse data types into a unified framework, thereby supporting personalized prevention strategies and informed clinical decision-making.

### BACKGROUND OF THE INVENTION

The prediction of individual disease risk is a fundamental goal in epidemiology and personalized medicine. Traditionally, models for disease risk prediction have focused on single genetic factors or family history to estimate an individual's susceptibility to complex diseases. With the advent of genome-wide association studies (GWAS), Polygenic Risk Scores (PRS) have emerged as a method to aggregate the effects of numerous genetic variants across the genome, providing a more comprehensive estimate of genetic predisposition to various diseases (Torkamani et al., 2018; Lewis & Vassos, 2020).

PRS have shown promise in predicting risks for conditions such as coronary artery disease, breast cancer, and type 2 diabetes (Inouye et al., 2018; Mavaddat et al., 2019; Läll et al., 2017). These scores are calculated by summing the number of risk alleles carried by an individual, each weighted by the effect size derived from GWAS data. While PRS can stratify individuals into different risk categories, they often explain only a modest proportion of disease heritability and may not capture the full genetic architecture of complex diseases (Wray et al., 2018; Chatterjee et al., 2016).

Environmental factors, including lifestyle behaviors such as diet, physical activity, and smoking, as well as socioeconomic status and exposure to environmental pollutants, significantly contribute to disease development (Pruss-Ustün et al., 2016; GBD 2017 Risk Factor Collaborators, 2018). For instance, tobacco smoking is a well-established environmental risk factor for lung cancer (Siegel et al., 2020), and poor diet combined with physical inactivity are major contributors to cardiovascular diseases and obesity (Yusuf et al., 2004). Studies have demonstrated that environmental exposures can modulate genetic risk and independently contribute to disease susceptibility (Manolio et al., 2009; Wild, 2005). Since environmental factors are often modifiable, they are critical targets for disease prevention and public health interventions.

The interplay between genetic predispositions and environmental exposures, referred to as gene-environment interactions, can substantially influence disease risk (Thomas, 2010; Hunter, 2005). Certain genetic variants may alter an individual's sensitivity to environmental risk factors. For example, individuals with specific genetic polymorphisms in detoxification enzymes may be more susceptible to the harmful effects of environmental toxins (Mahgoub et al., 1977). Recognizing and modeling these interactions are crucial for accurate disease risk prediction and for identifying high-risk individuals who may benefit from targeted interventions (Kraft & Hunter, 2009; Vineis & Pearce, 2020).

Despite the recognized importance of integrating genetic and environmental factors, existing predictive models often treat these elements separately, neglecting the complex interactions between them (Janssens & Joyner, 2019; Chatterjee & Wheeler, 2020). PRS models typically focus solely on genetic variants without incorporating environmental exposures, while environmental risk assessments may overlook genetic susceptibility. This siloed approach limits the predictive accuracy and clinical utility of these models (Amin-Naves et al., 2021).

Moreover, most PRS have been developed using data from individuals of European ancestry, reducing their applicability across diverse populations due to genetic heterogeneity (Martin et al., 2019; Peterson et al., 2019). Environmental exposures and their health impacts can also vary geographically and culturally, necessitating models that account for population-specific factors (Sirugo et al., 2019). There is a significant need for predictive models that can integrate genetic and environmental data, including their interactions, to enhance disease risk prediction and support personalized prevention strategies (Torkamani et al., 2017; Gallagher & Chen-Plotkin, 2018).

Recent research underscores the potential benefits of such integrated models. Studies have shown that using both PRS and environmental factors improves risk prediction for diseases like type 2 diabetes and breast cancer (Läll et al., 2021; Maas et al., 2016). The inclusion of gene-environment interactions further refines these predictions, providing a more nuanced understanding of disease risk (Wu et al., 2018).

However, integrating genetic and environmental data poses several challenges. Data heterogeneity is a significant obstacle, as genetic and environmental data are often collected and measured differently, complicating their integration (Vineis et al., 2013). Modeling the nonlinear and multiplicative interactions between numerous genetic variants and environmental factors requires sophisticated statistical methods (Li & Tsaih, 2020). Additionally, analyzing large-scale datasets with high-dimensional variables necessitates significant computational resources (Greene et al., 2014). Privacy and ethical considerations also play a crucial role, as handling sensitive genetic and personal data requires strict adherence to privacy regulations and ethical standards (Gymrek, 2017).

Advances in computational biology, machine learning, and data science are facilitating the integration of complex datasets (Shen et al., 2020; Rajkomar et al., 2019). Machine learning algorithms can handle high-dimensional data and uncover patterns and interactions that traditional statistical methods might miss (Abraham & Inouye, 2015). Additionally, the development of large biobanks and cohort studies that collect comprehensive genetic and environmental data provides a rich resource for developing integrated models (Bycroft et al., 2018).

In summary, there is a substantial gap in current disease risk prediction models due to the lack of integration of genetic and environmental factors and their interactions. Addressing this gap requires innovative methods and systems capable of combining diverse data types into a comprehensive risk assessment tool. The present invention aims to fulfill this need by providing a novel approach to calculate an integrated disease risk score. This approach enhances the accuracy and utility of disease risk predictions and ultimately contributes to better health outcomes through personalized medicine.

### SUMMARY OF THE INVENTION

The present invention provides a novel method and system for calculating an Integrated Risk Score (IRS) that holistically considers genetic predispositions, environmental exposures, and their interactions to enhance the accuracy of disease risk prediction. This integrated approach moves beyond traditional models that treat genetic and environmental factors separately, addressing the limitations of existing predictive tools.

The invention involves acquiring genetic information from individuals, including multiple genetic markers associated with specific diseases, and calculating a Polygenic Risk Score (PRS) based on the presence and effect sizes of risk alleles derived from genome-wide association studies (GWAS). It also gathers data on various environmental risk factors such as age, sex, geographic location, lifestyle behaviors (e.g., diet, physical activity, smoking status), socioeconomic status, and exposure to environmental pollutants. Both genetic and environmental data are processed and normalized to account for differences in measurement scales and units.

A weighted algorithm with interaction modeling is employed, where specific weights are assigned to each genetic marker and environmental factor based on their relative contributions to disease risk, as established by comprehensive epidemiological research. Interaction terms are included in the algorithm to model the complex interplay between genetic and environmental factors, capturing multiplicative and nonlinear effects on disease risk. The weights and interaction parameters can be dynamically updated based on new epidemiological data, enabling the model to reflect temporal and regional variations in disease risk.

By integrating the weighted genetic and environmental factors along with their interactions, the system computes the IRS, a composite score that quantitatively represents an individual's overall disease risk. Additionally, a confidence score is provided, reflecting the reliability of the risk assessment based on data completeness and the significance of contributing factors. This aids in interpreting the IRS and supports informed decision-making.

The system architecture includes a comprehensive database to store genetic and environmental data, weights, and interaction parameters, which is regularly updated with the latest research findings. Advanced computational models, including sophisticated statistical methods and machine learning algorithms, are employed to handle high-dimensional data and complex interactions. An intuitive user interface allows for secure data input and result retrieval, and the system can be implemented across various platforms, including mobile applications, web-based servers, and integrated modules within electronic health systems.

This invention has significant applications and use cases, such as assisting healthcare providers in identifying individuals at high risk for specific diseases, enabling personalized prevention strategies and early interventions. It empowers individuals with insights into their health risks, promoting proactive lifestyle changes and informed health decisions. Additionally, it informs public health policies and resource allocation by identifying population-level risk patterns and contributing factors.

The advantages of the invention include enhanced predictive accuracy by integrating genetic and environmental data, including their interactions, providing a more comprehensive and accurate assessment of disease risk compared to models that consider these factors separately. It offers personalization by tailoring risk assessments to the individual level, accounting for unique genetic makeups and environmental exposures. The model's adaptability allows it to be updated with new data and adapted to various diseases, populations, and geographic regions. Furthermore, it is user-friendly and accessible through multiple platforms with a focus on ease of use and data security.

### DETAILED DESCRIPTION OF THE INVENTION

The present invention provides a comprehensive method and system for predicting phenotypes of individuals by integrating genetic data, environmental factors, and their interactions. By leveraging advanced statistical analyses, machine learning techniques, and computational models, the invention enhances the accuracy of phenotype prediction beyond what is achievable by considering genetic or environmental factors alone. This integrated approach supports personalized healthcare strategies, research initiatives, and public health policies.

The invention involves collecting genetic and environmental data from individuals, processing this data using predictive algorithms that incorporate weights and interaction terms, and generating phenotype predictions along with confidence scores. The system architecture includes a database for data storage, a processor for computations, an output device for displaying results, and a user interface for data input and interaction. The system can be implemented across various platforms, such as mobile applications, web-based servers, or integrated modules within electronic health systems.

### Methodology for Phenotype Prediction

a. Genetic Data Collection and Analysis
Data acquisition: genetic data is obtained from individuals through methods such as genomic sequencing, genotyping arrays, or other molecular diagnostic techniques. The data includes multiple genetic markers (e.g., single nucleotide polymorphisms or SNPs) associated with phenotypes of interest.
Genetic score calculation: a genetic score is calculated by summing the effect sizes of alleles present in the individual's genome. Effect sizes are derived from genome-wide association studies (GWAS) or other genetic research. The genetic score formula is:

$$G = {\sum }_{k - 1}^{m} {β}_{k} \times {X}_{k}$$

Where:
   βₖ is the effect size of the h allele.
   Xₖ is the genotype of the individual at the k^{th} locus (e.g., 0, 1, or 2 copies of the allele).
   m is the total number of genetic markers considered.
Data normalization: The genetic score may be normalized to facilitate integration with environmental data.
   b. Environmental Data Collection and Processing
Data acquisition: environmental data is collected from various sources, including:
   Self-reported questionnaires: Information on lifestyle behaviors such as diet, physical activity, smoking status, alcohol consumption, stress levels, and sleep patterns.
   Electronic Health Records (EHRs): Medical history, comorbidities, medication use, clinical measurements, and laboratory results.
   Environmental Monitoring Systems: Data on air quality, pollution levels, climate variables, and exposure to environmental toxins.
   Geographic Information Systems (GIS): Geographic location data to assess regional environmental factors.
   Wearable devices: continuous monitoring of physiological parameters like heart rate, activity levels, and sleep quality.
   Socioeconomic data: information on income level, education, occupation, and access to healthcare.
Data Processing:
   Quantification: Environmental factors are quantified to convert qualitative data into numerical values where applicable.
   Normalization: data is normalized or standardized to account for differences in scale and measurement units.
   Encoding: categorical variables are encoded using techniques such as one-hot encoding or ordinal encoding to make them suitable for computational models.
   Data imputation: missing data is handled using imputation methods like mean substitution, regression imputation, or machine learning-based techniques.
      c. Predictive Algorithm Application
Weight Assignment:
   Regression analyses: Weights are determined through regression analyses (e.g., linear regression, logistic regression, Cox proportional hazards regression) applied to population-level data or epidemiological studies.
   Statistical modeling: Weights may also be derived from statistical models that assess the relative contribution of each factor to the phenotype.
   Expert consensus: In the absence of sufficient data, expert opinion may guide weight assignment.
Integration of data:
   Predictive algorithm: The genetic score and processed environmental data are integrated using a predictive algorithm that may include:
      Regression models: modeling the relationships between variables using regression techniques.
      Machine learning techniques: algorithms such as random forests, gradient boosting machines, neural networks, support vector machines, or ensemble methods to capture complex patterns and interactions.
      Statistical models: methods like Bayesian inference, principal component analysis, or cluster analysis to reduce dimensionality and identify significant factors.
   Interaction terms:
      Gene-environment interactions: incorporating terms that model the interaction between genetic markers and environmental factors.
      Environment-environment interactions: including interactions among environmental factors that may jointly influence the phenotype.
      Mathematical representation: interaction terms are represented mathematically to capture multiplicative or nonlinear effects.
         d. Phenotype Prediction
Prediction output:
   Quantitative phenotypes: predicting numerical values for traits such as blood pressure, cholesterol levels, or height.
   Qualitative phenotypes: classifying categorical outcomes like disease presence or absence, treatment response, or behavioral tendencies.
   Multi-phenotype prediction: simultaneously predicting multiple phenotypes that may be interrelated.
Confidence score calculation:
   Reliability assessment: A confidence score is calculated to reflect the reliability of the prediction based on factors such as data completeness, model performance metrics, and the statistical significance of contributing factors.
   Interpretability: Confidence scores aid in interpreting the predictions and making informed decisions.
      e. Output and interpretation
Results presentation:
   Visualization tools: graphs, charts, or other visual aids help users understand their predicted phenotypes and confidence scores.
   Reporting: detailed reports can be generated, summarizing the inputs, prediction results, and recommended actions.
Actionable insights:
   Personalized recommendations: Based on the predicted phenotypes, individuals may receive tailored advice on lifestyle modifications, preventive measures, or medical consultations.
   Clinical decision support: Healthcare providers can use the predictions to inform diagnosis, treatment planning, and patient management.

### System Architecture and Components

a. Database
Data storage:
   Genetic data repository: secure storage of genetic information, ensuring compliance with privacy regulations.
   Environmental data repository: storage of environmental factors, including longitudinal data to track changes over time.
   Phenotype data repository: accumulation of phenotype data for model training and validation.
Data management:
   Data integrity: regular data validation checks to maintain accuracy.
   Scalability: Infrastructure capable of handling large datasets and high user volumes.
      b. Processor and Computational Models
Computational capabilities:
   High-performance computing: utilization of powerful processors or cloud-based computing resources to handle complex calculations.
   Parallel processing: implementing parallel algorithms to improve efficiency.
Model development:
   Training and validation: using collected data to train predictive models and validate their performance using techniques like cross-validation.
   Model updating: periodic retraining of models with new data to enhance accuracy and adapt to emerging trends.
Software implementation:
   Programming Languages: Use of languages suitable for data science and machine learning (e.g., Python, R).
   Libraries and frameworks: Employing libraries such as scikit-learn, tensor flow, or pytorch for machine learning implementations.
      c. Output Device
User interface devices:
   Computers and laptops: access through web browsers or installed software.
   Mobile devices: Smartphones and tablets via mobile applications.
   Wearables: Integration with smartwatches or fitness trackers for real-time feedback.
Accessibility features:
   Multi-language support: catering to users from different linguistic backgrounds.
   User-friendly design: intuitive navigation and clear presentation of information.
      d. User Interface
Data input mechanisms:
   Manual entry: users can input data through forms or questionnaires.
   Automated data retrieval: integration with electronic health records or wearable devices for automatic data collection.
   Data verification: implementing checks to ensure data accuracy and completeness.
Privacy controls:
   Consent management: users can provide or withdraw consent for data use and sharing.
   Anonymization options: allowing users to anonymize their data for research purposes.
Educational resources:
   Information libraries: providing articles, videos, or interactive modules on genetics, environmental health, and phenotypes.
   FAQs and support: addressing common questions and offering assistance.

### Implementation Platforms

a. Mobile application: features:
   Notifications: alerts for new predictions, updates, or recommended actions.
   Data synchronization: real-time syncing with wearable devices and other data sources.
   User engagement: Gamification elements to encourage healthy behaviors.
b. Web-based server: features:
   Cross-platform compatibility: accessible from various devices and operating systems.
   High availability: ensuring minimal downtime with robust server infrastructure.
   Secure access: implementing HTTPS protocols and secure authentication methods.
c. Standalone software application: features:
   Customization: institutions can tailor the software to their specific needs.
   Integration: seamless incorporation into existing healthcare systems or research platforms.
   Offline functionality: ability to operate without continuous internet access.

### Applications and Use Cases

a. Clinical Settings
Personalized medicine: Tailoring treatments based on predicted drug responses or disease susceptibility.
Risk stratification: Identifying individuals at higher risk for certain conditions for early intervention.
Monitoring progress: Tracking phenotype changes over time to assess treatment efficacy.
   b. Personal Health Management
Lifestyle modification: encouraging changes in diet, exercise, or habits based on predicted phenotypes.
Preventive health: empowering individuals to take proactive steps in managing their health.
Family planning: providing insights into hereditary traits or conditions.
   c. Research and Public Health
Epidemiological studies: analyzing data at the population level to identify trends and risk factors.
Policy development: informing public health policies and resource allocation.
Genetic counseling: assisting professionals in advising patients based on comprehensive risk assessments.

### EXAMPLES AND SCENARIOS

### Example 1: Predicting Risk of Type 2 Diabetes

Genetic factors: SNPs associated with insulin resistance and pancreatic beta-cell function.
Environmental factors: BMI, diet, physical activity, family history, socioeconomic status.
Outcome: prediction of diabetes risk, personalized recommendations for prevention.

### Example 2: Pharmacogenomics

Genetic factors: variants affecting drug metabolism enzymes like CYP450.
Environmental factors: concurrent medications, alcohol use, liver function.
Outcome: prediction of drug efficacy and risk of adverse effects, guiding medication choices.

### Example 3: Athletic Performance Traits

Genetic factors: genes influencing muscle fiber composition, oxygen utilization.
Environmental factors: training regimen, nutrition, recovery practices.
Outcome: prediction of athletic potential, optimization of training programs.

### Advantages and Improvements Over Prior Art

Comprehensive integration: simultaneous consideration of genetic and environmental factors with their interactions.
Enhanced predictive accuracy: improved models capturing complex relationships, leading to better phenotype predictions.
Personalization: tailored insights and recommendations for individuals.
Adaptability: applicable to a wide range of phenotypes across different populations.
User engagement: interactive and educational interfaces that promote user involvement.

### Ethical and Privacy Considerations

Informed consent: clear communication about data use and obtaining consent.
Data security: robust encryption and security protocols to protect user data.
Transparency: providing users with explanations of how predictions are made.
Equity and accessibility: ensuring the system is accessible to diverse populations and does not exacerbate health disparities.

### Technical Implementation Details

Software architecture:
   Modular design: separation of data management, business logic, and presentation layers.
   APIs: application programming interfaces for integration with external systems.
Data processing pipelines:
   Preprocessing steps: data cleaning, normalization, and transformation.
   Model deployment: utilizing containers or virtual environments for scalability.
Performance optimization:
   Algorithm efficiency: choosing algorithms appropriate for the dataset size and complexity.
   Resource management: dynamic allocation of computing resources.

### Future Enhancements

Incorporation of additional data types: including epigenetic data, microbiome profiles, or social determinants of health.
Real-time analytics: providing immediate feedback based on continuous data streams.
Artificial intelligence explainability: implementing techniques to make complex models interpretable.

### REFERENCE

1. Abraham, G., & Inouye, M. (2015). Genomic risk prediction of complex human disease and its clinical application. Current Opinion in Genetics & Development, 33, 10-16.
2. Amin-Naves, J., Zoghbi, M., & Cordell, H. J. (2021). The challenges of gene-environment interaction analysis: lessons learned from asthma. Human Genetics, 140(1), 21-43.
3. Bycroft, C., Freeman, C., Petkova, D., et al. (2018). The UK Biobank resource with deep phenotyping and genomic data. Nature, 562(7726), 203-209.
4. Chatterjee, N., & Wheeler, B. (2020). Statistical and bioinformatics methods for studying the exposome and metabolome. Annual Review of Public Health, 41, 313-327.
5. Chatterjee, N., Shi, J., & Garcia-Closas, M. (2016). Developing and evaluating polygenic risk prediction models for stratified disease prevention. Nature Reviews Genetics, 17(7), 392-406.
6. Gallagher, M. D., & Chen-Plotkin, A. S. (2018). The post-GWAS era: from association to function. American Journal of Human Genetics, 102(5), 717-730.
7. GBD 2017 Risk Factor Collaborators. (2018). Global, regional, and national comparative risk assessment of 84 behavioral, environmental and occupational, and metabolic risks for 195 countries and territories. The Lancet, 392(10159), 1923-1994.
8. Greene, C. S., Tan, J., Ung, M., et al. (2014). Big data bioinformatics. Journal of Cellular Physiology, 229(12), 1896-1900.
9. Gymrek, M. (2017). Breaking down barriers: accessing genomic data to solve rare Mendelian diseases. PLoS Biology, 15(10), e2002365.
10. Hunter, D. J. (2005). Gene-environment interactions in human diseases. Nature Reviews Genetics, 6(4), 287-298.
11. Inouye, M., Abraham, G., Nelson, C. P., et al. (2018). Genomic risk prediction of coronary artery disease in 480,000 adults: implications for primary prevention. Journal of the American College of Cardiology, 72(16), 1883-1893.
12. Janssens, A. C., & Joyner, M. J. (2019). Polygenic risk scores that predict common diseases using millions of single nucleotide polymorphisms: is more, better? Clinical Chemistry, 65(5), 609-611.
13. Kraft, P., & Hunter, D. J. (2009). Genetic risk prediction—are we there yet? New England Journal of Medicine, 360(17), 1701-1703.
14. Läll, K., Magi, R., Morris, A., et al. (2017). Personalized risk prediction for type 2 diabetes: the potential of genetic risk scores. Genetics in Medicine, 19(3), 322-329.
15. Läll, K., Ripatti, P., Orrù, V., et al. (2021). Polygenic prediction of type 2 diabetes in a genetically homogeneous population. Diabetes, 70(1), 137-146.
16. Li, R., & Tsaih, S. W. (2020). Machine learning approaches for elucidating the genetic architecture of complex traits. Frontiers in Genetics, 11, 699.
17. Lewis, C. M., & Vassos, E. (2020). Polygenic risk scores: from research tools to clinical instruments. Genome Medicine, 12(1), 44.
18. Maas, P., Barrdahl, M., Joshi, A. D., et al. (2016). Breast cancer risk from modifiable and nonmodifiable risk factors among white women in the United States. JAMA Oncology, 2(10), 1295-1302.
19. Mahgoub, A., Idle, J. R., Dring, L. G., et al. (1977). Polymorphic hydroxylation of Debrisoquine in man. The Lancet, 310(8038), 584-586.
20. Manolio, T. A., Collins, F. S., Cox, N. J., et al. (2009). Finding the missing heritability of complex diseases. Nature, 461(7265), 747-753.
21. Martin, A. R., Kanai, M., Kamatani, Y, et al. (2019). Clinical use of current polygenic risk scores may exacerbate health disparities. Nature Genetics, 51(4), 584-591.
22. Mavaddat, N., Michailidou, K., Dennis, J., et al. (2019). Polygenic risk scores for prediction of breast cancer and breast cancer subtypes. American Journal of Human Genetics, 104(1), 21-34.
23. Peterson, R. E., Kuchenbaecker, K., Walters, R. K., et al. (2019). Genome-wide association studies in ancestrally diverse populations: opportunities, methods, pitfalls, and recommendations. Cell, 179(3), 589-603.
24. Prüss-Ustün, A., Wolf, J., Corvalán, C., et al. (2016). Preventing disease through healthy environments: a global assessment of the burden of disease from environmental risks. World Health Organization.
25. Rajkomar, A., Dean, J., & Kohane, I. (2019). Machine learning in medicine. New England Journal of Medicine, 380(14), 1347-1358.
26. Shen, X., Howard, D. M., Adams, M. J., et al. (2020). A phenome-wide association and Mendelian Randomisation study of polygenic risk for depression in UK Biobank. Nature Communications, 11(1), 2301.
27. Siegel, R. L., Miller, K. D., & Jemal, A. (2020). Cancer statistics, 2020. CA: A Cancer Journal for Clinicians, 70(1), 7-30.
28. Sirugo, G., Williams, S. M., & Tishkoff, S. A. (2019). The missing diversity in human genetic studies. Cell, 177(1), 26-31.
29. Thomas, D. (2010). Gene-environment-wide association studies: emerging approaches. Nature Reviews Genetics, 11(4), 259-272.
30. Torkamani, A., Andersen, K. G., Steinhubl, S. R., & Topol, E. J. (2017). High-definition medicine. Cell, 170(5), 828-843.
31. Torkamani, A., Wineinger, N. E., & Topol, E. J. (2018). The personal and clinical utility of polygenic risk scores. Nature Reviews Genetics, 19(9), 581-590.
32. Vineis, P., van Veldhoven, K., Chadeau-Hyam, M., & Athersuch, T. J. (2013). Advancing the application of omics-based biomarkers in environmental epidemiology. Environmental and Molecular Mutagenesis, 54(7), 461-467.
33. Vineis, P., & Pearce, N. (2020). Missing heritability in genome-wide association study research. Nature Reviews Genetics, 21(8), 573.
34. Wild, C. P. (2005). Complementing the genome with an “exposome”: the outstanding challenge of environmental exposure measurement in molecular epidemiology. Cancer Epidemiology Biomarkers & Prevention, 14(8), 1847-1850.
35. Wray, N. R., Wijmenga, C., Sullivan, P. F., et al. (2018). Common disease is more complex than implied by the core gene omnigenic model. Cell, 173(7), 1573-1580.
36. Wu, Y, Zeng, J., Zhang, F., et al. (2018). Integrative analysis of omics summary data reveals putative mechanisms underlying complex traits. Nature Communications, 9(1), 918.
37. Yusuf, S., Hawken, S., Ounpuu, S., et al. (2004). Effect of potentially modifiable risk factors associated with myocardial infarction in 52 countries (the INTERHEART study): case-control study. The Lancet, 364(9438), 937-952.

What is claimed:

1. A method for predicting phenotypes of an individual by integrating genetic and environmental data, the method comprising:
a. Obtaining genetic data from the individual, wherein the genetic data includes multiple genetic markers associated with one or more phenotypes of interest;
b. Calculating a genetic score based on the genetic data, wherein the genetic score is computed by summing effect sizes of alleles present in the individual's genome, each effect size derived from genome-wide association studies (GWAS) or other genetic research;
c. Obtaining environmental data associated with the individual, wherein the environmental data includes a plurality of environmental factors selected from the group consisting of age, sex, geographic location, lifestyle behaviors, socioeconomic status, and exposure to environmental elements;
d. Processing the environmental data by quantifying, normalizing, and encoding the environmental factors to facilitate integration with the genetic data;
e. Assigning weights to the genetic data and the environmental data based on their relative contributions to the phenotypes, wherein the weights are determined from epidemiological studies, expert consensus, or derived through statistical analyses such as regression analyses;
f. Applying a predictive algorithm that integrates the genetic score and the processed environmental data, the algorithm utilizing methods selected from regression analyses, statistical modeling, machine learning techniques, or combinations thereof, and incorporating interaction terms that model multiplicative and nonlinear effects between genetic markers and environmental factors;
g. Predicting the phenotype(s) of interest for the individual using the predictive algorithm, wherein the prediction may be a quantitative value or a qualitative classification;
h. Generating a confidence score associated with the phenotype prediction, wherein the confidence score reflects the reliability of the prediction based on data completeness and the significance of the contributing factors; and
i. Outputting the predicted phenotype(s) and the confidence score to an output device for presentation to the individual or a healthcare provider.

2. The method of claim 1, wherein the phenotypes include, but are not limited to, disease risk, physiological traits, behavioral characteristics, response to treatment, or any other measurable or observable characteristic influenced by genetic and environmental factors.

3. The method of claim 1, wherein the predictive algorithm comprises regression analyses selected from linear regression, logistic regression, Cox proportional hazards regression, generalized linear models, or multivariate regression techniques to model the relationships between genetic data, environmental data, and phenotypes.

4. The method of claim 1, wherein the predictive algorithm further comprises machine learning methods selected from random forests, gradient boosting machines, neural networks, support vector machines, decision trees, or ensemble methods to enhance prediction accuracy.

5. The method of claim 1, wherein the environmental data further includes data obtained from self-reported questionnaires, electronic health records, environmental monitoring systems, geographic information systems, and wearable devices.

6. The method of claim 1, wherein the weights assigned to the genetic data and environmental data are determined through regression coefficients obtained from regression analyses applied to population-level data or epidemiological studies.

7. The method of claim 1, wherein the interaction terms in the predictive algorithm model gene-environment interactions and environment-environment interactions that contribute to the phenotypes.

8. The method of claim 1, further comprising integrating additional data types selected from microbiome profiles, epigenetic markers, proteomic data, metabolomic data, or other biological markers to enhance the accuracy of the phenotype prediction.

9. The method of claim 1, further comprising:
a. Collecting comprehensive genetic and environmental data from a plurality of individuals;
b. Applying regression analyses to the collected data to determine the relative contributions of genetic markers and environmental factors to various phenotypes, and to identify significant interaction effects;
c. Training machine learning models on the collected data to capture nonlinear relationships and complex interactions that regression analyses may not fully address;
d. Validating the predictive models using statistical techniques and cross-validation methods to ensure predictive accuracy and generalizability;
e. Applying the trained models to new individual data to predict phenotypes and calculate associated confidence scores; and
f Updating the models periodically with new data to improve performance and adapt to emerging factors or population changes.

10. The method of claim 1, wherein the phenotypes include physiological traits, behavioral characteristics, disease susceptibility, treatment response, or any other measurable or observable characteristic influenced by genetic and environmental factors.

11. A system for predicting phenotypes of an individual by integrating genetic and environmental data, the system comprising:
a. A database configured to store genetic data, environmental data, weights, interaction parameters, and user information, wherein the database is secure, scalable, and regularly updated with new research findings;
b. A processor configured to:
i. Obtain genetic data and environmental data associated with the individual;
ii. Calculate a genetic score based on the genetic data;
iii. Process the environmental data by quantifying, normalizing, and encoding the environmental factors;
iv. Assign weights to the genetic data and environmental data based on their relative contributions to the phenotypes, utilizing methods such as regression analyses or statistical modeling;
v. Apply a predictive algorithm that integrates the genetic score and processed environmental data, the algorithm utilizing methods selected from regression analyses, statistical modeling, machine learning techniques, or combinations thereof, incorporating interaction terms;
vi. Predict the phenotype(s) of interest and calculate an associated confidence score;
c. An output device configured to display the predicted phenotype(s) and confidence score, along with visual aids to assist in interpretation; and
d. A user interface configured to allow secure input of genetic and environmental data, provide guidance on data entry, and present educational resources regarding phenotypes, risk factors, and recommendations.

12. The system of claim 11, further comprising:
a. A user interface that provides individuals with insights into their predicted phenotypes, generated using predictive algorithms that include regression analyses and other methods;
b. A feedback mechanism that allows users to input changes in environmental factors or lifestyle behaviors, with the system updating the phenotype predictions accordingly using updated regression analyses and predictive models;
c. An analytics module that tracks changes in predicted phenotypes over time, utilizing regression analyses and statistical methods to identify trends or patterns in the individual's data; and
d. A communication module that facilitates sharing of phenotype predictions and recommendations with healthcare providers or support networks, subject to user consent.

13. The system of claim 11, wherein the phenotypes include quantitative traits such as height, blood pressure, cholesterol levels, or glucose levels, and qualitative traits such as disease presence or absence, behavioral tendencies, or response to medications.

14. The system of claim 11, wherein the processor employs regression analyses selected from linear regression, logistic regression, Cox proportional hazards regression, generalized linear models, or multivariate regression techniques to model the relationships between variables and predict phenotypes.

15. The system of claim 11, wherein the processor employs machine learning algorithms selected from random forests, gradient boosting machines, neural networks, support vector machines, decision trees, or ensemble methods to enhance prediction accuracy.

16. The system of claim 11, wherein the processor utilizes statistical modeling techniques such as Bayesian inference, principal component analysis, or cluster analysis to identify patterns and relationships in the data.

17. The system of claim 11, wherein the predictive algorithm dynamically updates the weights and interaction parameters based on new data inputs, regression analyses, machine learning feedback, or updates from epidemiological research to reflect temporal, regional, or population-specific variations in phenotypes.

18. A non-transitory computer-readable medium containing instructions which, when executed by a processor, cause the processor to perform operations comprising:
a. Retrieving genetic data and environmental data associated with an individual;
b. Calculating a genetic score based on the genetic data;
c. Processing the environmental data by quantifying, normalizing, and encoding environmental factors;
d. Assigning weights to the genetic data and environmental data based on their relative contributions to the phenotypes, utilizing regression analyses or statistical modeling;
e. Applying a predictive algorithm that integrates the genetic score and processed environmental data, incorporating interaction terms and utilizing methods such as regression analyses, machine learning techniques, statistical modeling, or combinations thereof,
f. Predicting the phenotype(s) of interest, which may be quantitative values or qualitative classifications;
g. Generating a confidence score associated with the phenotype prediction, reflecting the reliability of the prediction; and
h. Outputting the predicted phenotype(s) and the confidence score for presentation on an output device.

19. The non-transitory computer-readable medium of claim 18, wherein the instructions cause the processor to employ regression analyses selected from linear regression, logistic regression, Cox proportional hazards regression, generalized linear models, or multivariate regression techniques to model the relationships between variables and predict phenotypes.

20. The non-transitory computer-readable medium of claim 18, wherein the instructions enable the processor to incorporate machine learning algorithms or statistical modeling techniques to enhance prediction accuracy and identify complex patterns in the data.
